(19) United States
(12) Patent Application Publication
(10) Pub. No.: US 2007/0225783 A1
(43) Pub. Date: Sep. 27, 2007

(54) PAIN RELIEF APPARATUS

(76) Inventors: Aurelia Koby, San Diego, CA (US); Ian Macmorran, San Diego, CA (US)

Correspondence Address: KLEIN, O'NEILL & SINGH, LLP, 43 CORPORATE PARK, SUITE 204, IRVINE, CA, 92606, US

(21) Appl. No.: 11/277,475

(22) Filed: Mar. 24, 2006

(51) Int. Cl. A61F 7/00

(52) U.S. Cl. 607/108; 607/110; 607/112; 607/114

(57) ABSTRACT

A pain relief apparatus has an elongated fabric envelope or sleeve and at least one thermal pack located within a central portion of the sleeve. The thermal pack is a flexible plastic pouch containing a thermal material having substantial thermal retention. The envelope has at least one opening for receiving the thermal pack. The fabric envelope terminates at opposite ends in elongated end ties for encircling a body part and retaining the envelope and enclosed thermal pack in contact with the body part by interlacing or tying the ends of the end ties.

### PAIN RELIEF APPARATUS

This invention relates to the field of pain relief. More specifically, this invention relates to an apparatus, a system and a method for a portable pain relief device. Further, the invention relates to a versatile wrap and insertion pack that may be used to either heat and/or cool a particular location of an individual to provide temporary relief of minor pain. Additionally, the apparatus may be used in a plurality of different configurations to be adapted for use in a plurality of different locations on the individual.

### BACKGROUND OF THE INVENTION

The industry of pain relief has become a multi-million dollar industry. Medicines of all kinds are produced and marketed to relieve pain and to prevent further injury. As individuals get older, their bodies begin to break down and certain parts of their bodies tend to have become prone to pain. Additionally, those individuals that are active, such as athletes, amateur recreational participants and even children may have injuries from over strenuous use of their bodies. Another major area where pain relief is often necessary is that of repetitive motion or soft tissue injuries which often arise from repeated use of computer keyboards and the like, or any other repeated motion.

One area of the body that is prone to injury more than most other structural elements of the body is the muscles. In certain activities, the muscles may become tight, overused, stretched or bruised. When muscles are injured in some way, they tend to be painful to the touch and can cause significant discomfort in the individual with the injury. Additionally, stress in an individual may produce similar feelings of muscle injury in an individual because usually in times of high stress, an individual tends to be very tense, causing the muscles to tense. The muscle tenses and after a time, the muscles become painful.

Similarly, athletes or amateur individuals that practice strenuous physical exercise, or individuals who carry out repetitive movements in their work or other activities, may face problems with muscles that are sore, tender, swollen, or otherwise painful. The need for some relief becomes necessary in a lot of cases. There were two accepted principles to soothe muscle pain. The first principle was to chill, or ice a swollen muscle, which would decrease the amount of swelling in a muscle or joint. However, a problem with the direct application of ice is that the skin can become very cold and can cause the individual discomfort. For this reason, many individuals will wrap the ice in a cloth and apply to the area of discomfort. However, without provision for retaining moisture and for easily securing the cloth in position on the effected body part the overall effect is compromised by the wetness of the melting ice and the difficulty of maintaining the wrapped ice in position over the effected area. Another problem with this type of pain relief is that the individual had to hold the ice bag in place in order to keep swelling down or to continue pain relief. Therefore the individual was restricted in what activities could be performed during pain relief. Typically, an individual would have to sit in one place and hold the ice in direct contact with the affected area.

Cold has proven to be useful with many conditions, such as migraine, multiple sclerosis, sciatic nerve pain, sport injuries, and pain and swelling after surgery or dental treatment. For example a cold pack on the back of the neck will help reduce the swollen blood vessels and regulate the blood flow to the brain in a migraine attack. Generally, cold is of great benefit in headache relief. Scientific research also suggests a link between cooling to symptom improvement in MS patients. Research has shown cooling therapy can improve vision, movement, muscle strength and coordination. Cold therapy is suggested by doctors to be applied directly to the lower back area to relieve sciatic nerve pain. The cold will help reduce the swelling. Once the swelling has gone down, heat can be applied to increase circulation. After surgery or dental work, deep tissue cooling of injured areas is recommended to reduce pain and muscle spasms, reduce swelling, and help prevent further tissue damage.

The second principle of pain relief is the use of a hot pad to relieve muscle tightness and knotting. The hot pad can be placed on the outer surface of the skin of the individual in contact with the affected muscles/joints to soothe the muscle and/or joints. However, the same limitations are involved in this process as were described above. The user is similarly limited in mobility and activities that could be performed during the use of this hot pad and/or other similar device. The individual is limited to applying the hot pad directly to the affected area and cannot perform other activities during the time that the individual requires pain relief.

Heat improves circulation, improves cell function (metabolism), decreases stiffness in tendons and ligaments, relaxes the muscles and decreases muscle spasm, and lessens pain. Medical professionals explain that heat causes dilation of the blood vessels in the area being treated. This increase in blood flow brings fresh blood to the area and takes waste away from it. The result is that heat eases pain and speeds healing. Fibromyalgia pain responds very well to heat. Heat applied to sore muscles can provide immediate drug-free relief. Heat can relax muscles and stimulate blood circulation. Another option is to use cold to numb the sore area and reduce inflammation and swelling. This is especially good for joint pain caused by an arthritis flare-up. Heat can also be used in cases of TMJ syndrome. Heat is applied to the affected side(s) of the face. Although heat is most effective for many TMJ sufferers, cold may work better in some instances. Menstrual cramps can also be alleviated by application of heat. Applying heat to the cramped area will help promote blood flow to the muscles and relax the spasms. Heat is also sometimes used as a lactation aid for new mothers. Applying a warm surface to the breast will help the “let down” reflex. Warmth will help relax and soothe a baby with colic to prepare them for sleep. Another area where heat application can be useful is to ease growing pains in knees and calves of children and adolescents.

A need therefore exists for an improved apparatus and device to provide pain relief for repetitive stress injuries, sports injuries, or pain arising from other causes. Additionally, a need exits for an improved apparatus and device that may be used by an individual that provides long term pain relief to aches and pains, muscles, joints, tendons and the like. Moreover, a need therefore exists for an improved apparatus that may be easier to use and efficient for the purpose it is used for. Further, a need therefore exists for an apparatus that can have both heating properties for soothing and relieving tension and cooling properties for alleviating swelling and the like.

### SUMMARY OF THE INVENTION

It is an object of the present invention to provide a new and improved apparatus, a system and a method for a pain relief device. More specifically, the present invention provides an apparatus, system and method for pain relief device that is simple to use, efficient and has both heating and cooling functions. Additionally, the present invention provides an apparatus, a system and a method for a pain relief device wherein the device can be configured in a plurality of different positions and at different locations on the individual for use without excessive manipulation by the end user.

According to one aspect of the present invention, a pain relief apparatus is provided, which comprises an elongated fabric envelope, a thermal pack comprising at least one flexible plastic pouch containing a thermal material having substantial thermal retention, said envelope having at least one opening for receiving said thermal pack, said fabric envelope terminating at opposite ends in elongated end ties for encircling a body part and retaining said envelope in contact with the body part by interlacing the ends of said end ties.

The thermal pack may be heated or cooled prior to insertion in the envelope, so as to use heat or cold for pain relief and the like. When heated, the device may be used to heat and relax aching or stiff muscles. The apparatus acts as a thermal wrap which may be applied to sore muscles to provide immediate drug-free relief.

### BRIEF DESCRIPTION OF THE DRAWINGS

The present invention will be better understood from the following detailed description of an exemplary embodiment of the invention, in which like reference numerals refer to like parts, and in which;

FIG. 1 is a perspective view of the pain relief apparatus showing it extended prior to use;

FIG. 2 is a top plan view of the pain relief apparatus of the present invention showing the slit provided to insert and remove a thermal pack;

FIG. 3 is sectional view taken along line 3-3 of FIG. 2 and illustrates an embodiment of the pain relief apparatus showing the envelope of the apparatus enclosing a thermal pack which in this embodiment is a gel pack;

FIG. 4 is a perspective view of the present invention showing the pain relief apparatus positioned on the forehead of a user;

FIG. 5 is a perspective view of the pain relief apparatus of the present invention showing how the end ties may be positioned to secure the apparatus on the instep of the foot of a user,

FIG. 6 is a partial side view of the pain relief apparatus showing the apparatus secured over the knee of a user;

FIG. 7 is a side view of the pain relief apparatus showing the apparatus secured over the sole of a user's foot; and

FIG. 8 is a side view of pain relief apparatus showing the apparatus secured over the wrist of a user.

### DETAILED DESCRIPTION OF THE INVENTION

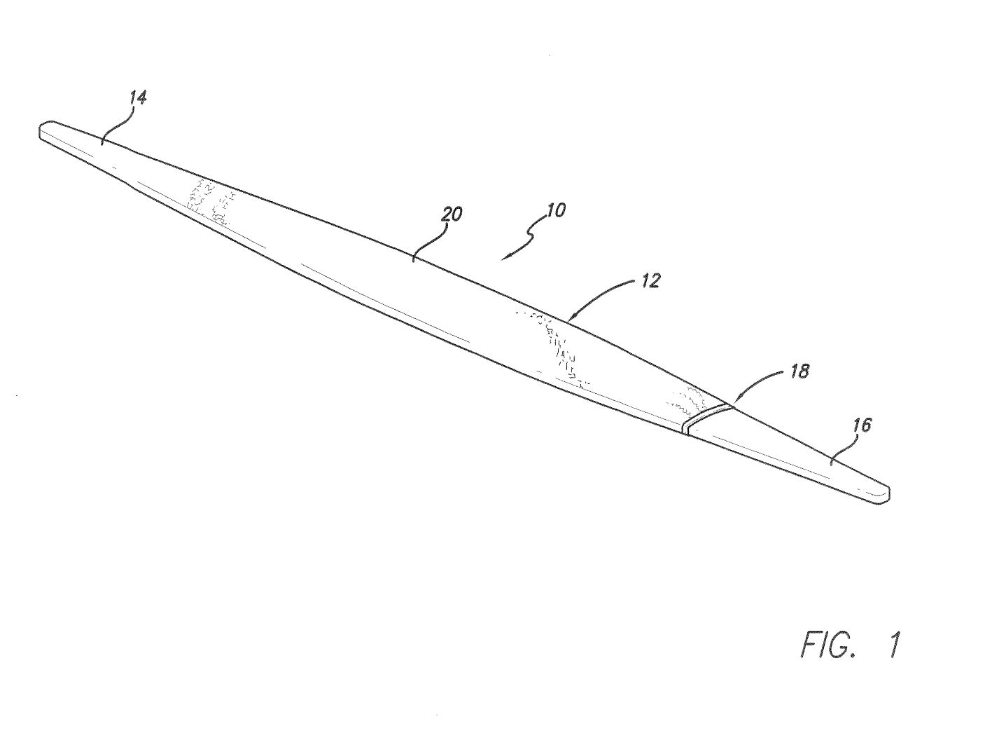

Referring to FIGS. 1 to 3, there is illustrated the pain relief apparatus 10, as it appears when extended and supported from a flat surface. The apparatus a central fabric envelope 20 and two fabric end ties 14 and 16. The fabric is a soft cotton based elasticized material. The use of a cotton blend is preferred and in the preferred embodiment the blend is approximately 90% cotton and 10% stretch fiber such as polyester or spandex. A cotton blend is preferred because of the characteristics of that natural fiber including breathability and absorbency. Polyester or other modern fiber is used to introduce increased stretchability to the fabric. Stretchability in the case of the preferred embodiment is defined as the capability of the fabric to accommodate a lengthwise extension of the envelope and end ties of at least 30%. This stretchability produces the capability of the apparatus to comfortably conform to the shape of virtually any part of the body. The preferred embodiment of the apparatus is approximately 36 inches in length and yet the stretchability of the apparatus allows it to be extended around and tie, for example over the 42 inch chest area on a user, such as to apply heat or cold to the user's upper back.

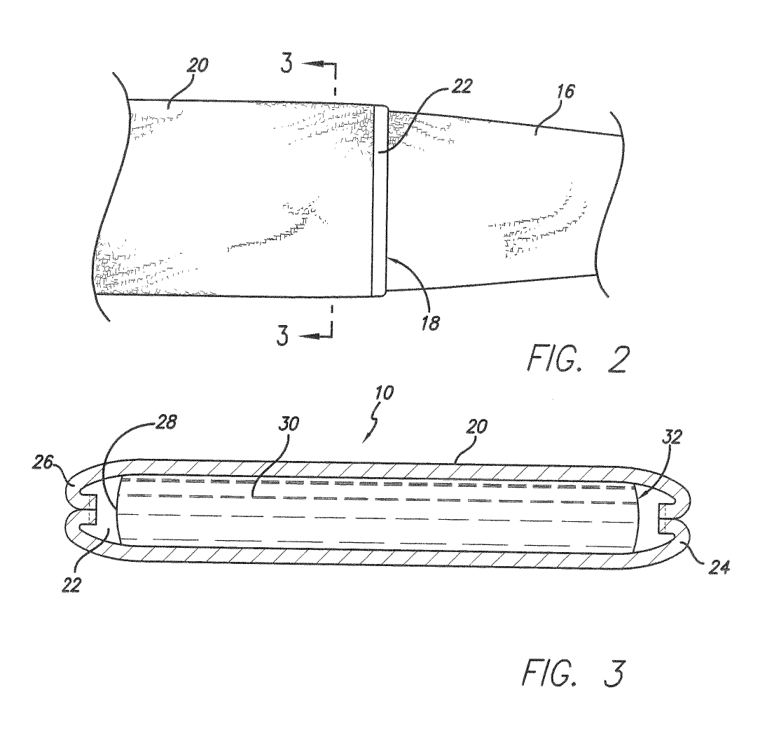

FIGS. 1 and 2 also illustrate the slit 18, which is formed by an opening in the envelope 20 with a fabric overlap. The slit allows the insertion and removal of a thermal pack (the thermal pack will be described in greater detail in reference to subsequent views). The overlap prevents inadvertent loss of the thermal pack as the apparatus is manipulated.

FIG. 1 shows that the envelope 20 tapers as it merges with the end tie 16. The slit 18 is shown to comprise an overlap of material 22 which may be finished by stitching (not shown).

FIG. 3 shows a thermal pack 32 inside envelope or sleeve 20. In the preferred embodiment this thermal pack extends longitudinally the entire longitudinal extent of the envelope 20. The thermal pack is prevented from entering the end ties 16 or 14 by selecting a thermal pack with a width that fits within the envelope 20 but not within the tapered sections that form the end ties. This construction avoids the necessity for sealing off the end ties and thereby avoids the seam or obstruction that could potentially form an undesirable ridge that might put pressure on the skin of the user. However, in alternative embodiments, the envelope and end ties may be of uniform width along the length of the device, with no taper. In this case, internal stitching or the like may be provided to retain the thermal pack in a central position within the envelope 20.

FIG. 3 illustrates the generally flattened cross sectional shape of the envelope with opposite side seams 24, 26 and an internal cavity 22 where the gel pack 32 is located in use. The envelope may be formed by two flat elongate pieces of material sewn together along the opposite side seams, as indicated in FIG. 3, or one elongate piece of material folded in half lengthwise and sewn together along one side seam. The gel pack 32 has a plastic film exterior 28 that encloses one or more compartments filled with thermal gel. The thermal gel selected for use with the invention has a high thermal capacity. As used herein a high thermal capacity refers to the characteristic of the material that allows a substantial amount of heat to be added or removed from the gel, producing a temperature differential from ambient and from the user's skin temperature. This high thermal capacity means that the gel and therefore the fabric envelope will retain a temperature differential for a substantial time after being brought into thermal contact with the user. The temperature differential causes heat to flow into or from the users tissues and thereby creates the desired therapeutic effect. The heat capacity and the quantity of the gel is selected so that substantial temperature differentials are achieved over at least 15 to 30 minutes when in thermally conductive contact with the user. One such thermal gel is a mixture of CMC (sodium carboxymethyl cellulose), propylene glycol, and distilled water, but any suitable thermal gel material may be used. The use of thermal gel is preferred over other thermal materials because the gel is reusable and remains pliable over a wide range of temperatures, including temperatures that would produce a change of state from liquid to solid in pure water. However, other thermal materials may be used in the practice of the invention. Other materials may include solids and heat producing materials such as iron particles that produce heat when exposed to air (oxygen).

Subject to precautions that would be included on packaging for the apparatus, the thermal pack can be heated or cooled in a variety of ways. The thermal pack may be removed for heating or cooling, which also permits the use of multiple packs so that one is being heated or cooled while the other is in use. When removed the pack can be heated in hot (warm) water. The pack can also be heated without removal from the envelope in a microwave oven and can be cooled with or without removal in the freezer section of a refrigerator. Of course, the pack should not be heated to temperatures that would cause a burn nor cooled to temperatures that could cause freezing necrosis of the skill or underlying tissue. The fabric envelope can be easily cleaned as necessary, simply by removing the gel pack and machine washing the envelope.

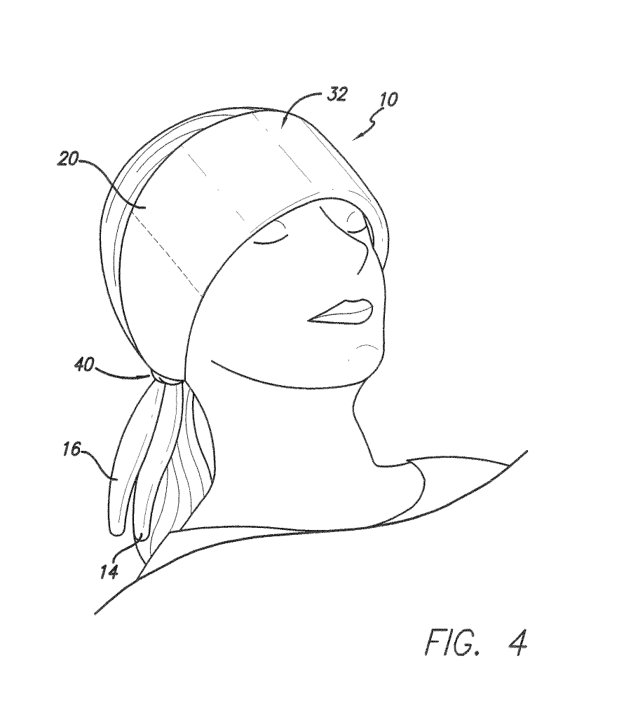

FIG. 4 shows the use of the apparatus 10 in application to the forehead of a user. The envelope 20 is centered lengthwise over the forehead. This in turn centers the thermal pack 32 over the forehead (the thermal pack is shown in dotted lines). As used for migraine or other headache the thermal pack would normally be chilled. The end ties 14 and 16 are shown draped behind the neck after being tied together in a knot 40. In some instances, it may not be necessary to complete a knot to hold the apparatus in position. Merely interlacing the end ties with one overlap may be sufficient because the elasticity can frictionally hold the end ties together and the envelope in contact with the body part.

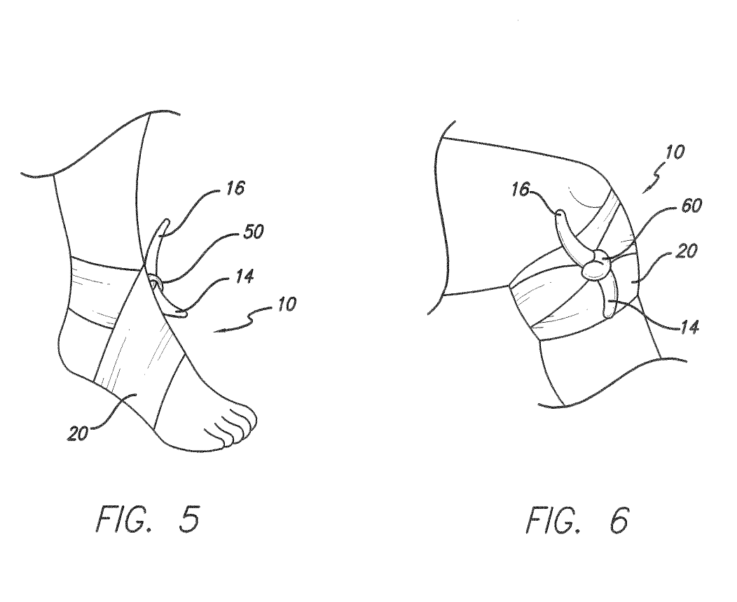

FIG. 5 shows the apparatus 10 secured to the foot of the user. In this example the envelope 20 and therefore the associated thermal pack (not shown) are positioned over the user's instep. The excess material is used to wrap the apparatus around the user's ankle before terminating in a knot 50 off to the side of instep. This positioning of the apparatus is a further demonstration of the versatility of the apparatus which allows it to be secured over a substantially any part of the user's anatomy. In the present example the user can apply slightly more tension to one end tie than the other resulting in the off-center knot 50 which therefore does not put pressure on the sore area of the instep.

FIG. 6 shows the apparatus 10 applied over the knee area of the user. The envelope 20 is centered over the frontal knee area to sooth a snore knee cap structure or other feature of the frontal portion of the lower knee. The end ties are used as described above to produce an off-center knot 60 which avoids putting pressure on the frontal portion of the knee.

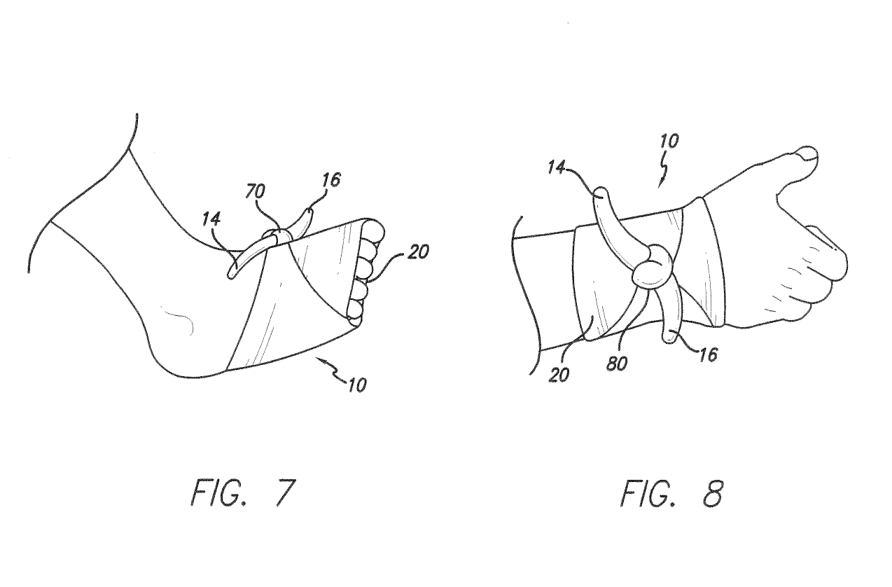

FIG. 7 shows the apparatus 10 position over the foot of the user with the envelope 20 centered over the ball of the users foot. The extra length of the envelope and end ties that are not needed to encircle the foot are consumed by a second wrap around the lower foot and terminated in a knot 70.

FIG. 8 shows an installation similar to that in FIG. 7 but this time positioned over the user's wrist to bring the thermal pack into heat conducting contact with the back of the user's wrist. As shown in FIG. 8 the end ties 14 and 16 are terminated in a knot 80. In this instance the knot is located directly over the effected area. This configuration may be desirable when it is desired to press the thermal pack into more direct heat conductive contact and where the resulting pressure does not cause discomfort. This might be the case, for example, where the injury is a deep tissue injury and it is desired to concentrate the thermal transfer directly over the injury to maximize heat transfer to bring maximum heating or cooling isolated to the effected area.

Although FIGS. 4 to 8 of the drawings illustrate the use of the pain relief device on various areas of the body, it will be understood that the device may be used in a similar manner anywhere on the body, such as the back, abdomen, elbow, neck, or the like, simply by holding the thermal pack against the area in question and then tying the end ties around the body, arm, neck, or the like, as appropriate. Longer end ties may be provided in devices for application around areas of the body with larger circumferences, such as the abdomen, back or the like.

It should be understood that various changes and modifications to the presently exemplary embodiments described herein will be apparent to those skilled in the art. Such changes and modifications may be made without departing from the spirit and scope of the present invention, which is defined by the appended claims.

What is claimed is:

1. Pain relief apparatus, comprising:
an elongated fabric envelope,
a thermal pack comprising at least one flexible plastic pouch containing a thermal material having substantial thermal retention,
said envelope having at least one opening for receiving said thermal pack,
said fabric envelope terminating at opposite ends in elongated end ties for encircling a body part and retaining said envelope in contact with the body part by interlacing the ends of said end ties.

2. The pain relief apparatus of claim 1, further comprising:
said opening comprising a crosswise slit in said fabric.

3. The pain relief apparatus of claim 2, wherein:
said slit is closed by an overlap of fabric extending over said opening and releaseably closing said slit.

4. The pain relief apparatus of claim 1, wherein:
said thermal material is a thermal gel.

5. The pain relief apparatus of claim 1, wherein said fabric has a relaxed length and is elasticized to extend lengthwise from the relaxed length to an extended length upon the application of hand tension to the ends of said envelope.

6. The pain relief apparatus of claim 5, wherein the extension from the relaxed length to the extended length is at least 30%.

7. The pain relief apparatus of claim 5, wherein:
said end ties comprise a continuation of the fabric of said envelope and are comprised of the same elasticized fabric.

8. The pain relief apparatus of claim 7, wherein said end ties are sized to exclude the entry of said pouch into the end ties.

9. A pain relief apparatus, comprising:
an elongated sleeve of fabric material, the sleeve having a central portion, opposite closed ends, and tie portions extending from opposite ends of said central portion up to said closed ends;
the sleeve being generally flattened and having opposite walls;
one of said walls having an opening; and
at least one thermal pack for insertion through said opening into the central portion of said sleeve, the thermal pack comprising a pouch of flexible, leak-proof material containing a thermal material having substantial thermal retention, and having a length substantially equal to the length of said central portion with opposite ends terminating short of said tie portions when said thermal pack is inserted into said sleeve;
whereby said central portion of said sleeve containing said thermal pack can be placed in contact with a body part and said tie portions comprise means for wrapping around said body part and tying together such that said apparatus encircles the body part and said central portion is secured in contact with the body part.

10. The apparatus as claimed in claim 9, wherein the central portion has a first, uniform width along its length, and each tie portion is of tapering width from the central portion up to the respective closed end.

11. The apparatus as claimed in claim 9, wherein the sleeve is made from an elasticized fabric material.

12. The apparatus as claimed in claim 11, wherein the fabric is a mixture of cotton and stretch fiber material.

13. The apparatus as claimed in claim 12, wherein the fabric is a mixture of 90% cotton and 10% spandex.

14. A method of applying heat or cold to a body part, comprising the steps of:
positioning a flexible thermal pack containing a thermal material at a predetermined temperature within a central portion of an elongated sleeve of fabric material;
positioning the central portion of the sleeve containing the thermal pack against a region of a body part to be heated or cooled; and
wrapping tie portions extending from opposite ends of the central portion of the sleeve about the body part and tying the ends of the tie portions together so as to secure the central portion and thermal pack against the body part.
